(19) United States
(12) Patent Application Publication
(10) Pub. No.: US 2025/0258117 A1
(43) Pub. Date: Aug. 14, 2025

(54) SYSTEM AND METHOD FOR QUANTIFYING THERMAL CONDUCTIVITY OF SUBSURFACE FORMATIONS FOR GEOTHERMAL ENERGY SYSTEMS

(71) Applicant: Chevron U.S.A. Inc., San Ramon, CA (US)

(72) Inventors: Jozina Belinda DIRKZWAGER, Houston, TX (US); Haijing WANG, Sugar Land, TX (US); Jeffrey Allen NUNN, Houston, TX (US); Jon Edward BURGER, Houston, TX (US)

(21) Appl. No.: 18/442,075

(22) Filed: Feb. 14, 2024

(51) Int. Cl. G01N 25/18; E21B 49/02; G01N 33/24

(52) U.S. Cl. CPC G01N 25/18; E21B 49/02; G01N 33/24

(57) ABSTRACT

A method is described for assessing subsurface formations for their suitability for use as an advanced close loop geothermal energy system by quantifying thermal conductivity of the subsurface formations. The thermal conductivity is quantified by obtaining rock samples and well logs from a well; analyzing the well logs to generate a well log interpretation; analyzing the rock samples to generate a rock sample interpretation; integrating the well log interpretation and the rock sample interpretation to generate a combined interpretation; estimating thermal conductivity along the well based on the combined interpretation using anisotropic equivalent media mixing laws; upscaling the thermal conductivity along the well to quantify thermal conductivity for the well as part of an advanced closed loop geothermal system; and quantifying the thermal conductivity of the subsurface formation based on the thermal conductivity for the well as part of an advanced closed loop geothermal system.

### CROSS-REFERENCE TO RELATED APPLICATIONS

Not applicable.

### STATEMENT REGARDING FEDERALLY SPONSORED RESEARCH OR DEVELOPMENT

Not applicable.

### TECHNICAL FIELD

The disclosed embodiments relate generally to techniques for assessing subsurface formations as geothermal systems. In particular, the techniques quantify the thermal conductivity of the subsurface formation to assess it for an Advanced Closed Loop (ACL) geothermal energy system.

### BACKGROUND

Geothermal energy systems can generate continuous and reliable electric power with near-zero emissions. Traditional geothermal systems require three key ingredients: heat, natural fractures, and fluid. Natural fractures near a heat source allow fluid to bring the heat from its source to the surface. However, traditional geothermal systems only exist in certain locations throughout the world, limiting its widespread deployment. As a result, geothermal energy usage has been low due to these limitations.

Advanced Closed Loop (ACL) systems overcome these limitations by drilling one or more wellbores through hot rocks and circulating a working fluid through the wellbores to absorb and transport heat to the surface for direct heating and electricity generation (Ramey, 1962; Horne, 1980; White et al., 2024). In ACL, fluid remains in the wellbores and heat transfer occurs conductively from hot rocks to the working fluid in the wellbores. In theory, ACL can be deployed in much broader geological locations to produce power from hot rock formations. Reservoir simulation is not required as the working fluid remains in the closed-loop system, limiting the risks of induced seismicity and lowers water consumption. There are many remaining challenges including complex downhole completions and sufficient heat transfer from the surrounding rock. One of the key parameters that affect the feasibility and performance of geothermal systems is the thermal conductivity of the rocks, which determines how efficiently heat can be transferred from the hot source to the production wells. Therefore, accurate and reliable methods and workflows to quantify the thermal conductivity of subsurface formations are essential for advanced geothermal systems exploration and assessment.

There exists a need for assessing subsurface formations for their suitability for ACL, thereby enabling improved geothermal energy production.

### SUMMARY

In accordance with some embodiments, a method for assessing subsurface formations for their suitability for use as an advanced close loop geothermal energy system by quantifying thermal conductivity of the subsurface formations is disclosed. The thermal conductivity is quantified by obtaining rock samples and well logs from a well; analyzing the well logs to generate a well log interpretation; analyzing the rock samples to generate a rock sample interpretation; integrating the well log interpretation and the rock sample interpretation to generate a combined interpretation; estimating thermal conductivity along the well based on the combined interpretation using anisotropic equivalent media mixing laws; upscaling the thermal conductivity along the well to quantify thermal conductivity for the well as part of an advanced closed loop geothermal system; and quantifying the thermal conductivity of the subsurface formation based on the thermal conductivity for the well as part of an advanced closed loop geothermal system. The thermal conductivity of the subsurface formation may be used to design well paths for an advanced closed loop geothermal system, drilling wells following the well paths, and building the advanced closed loop geothermal system.

In another aspect of the present invention, to address the aforementioned problems, some embodiments provide a non-transitory computer readable storage medium storing one or more programs. The one or more programs comprise instructions, which when executed by a computer system with one or more processors and memory, cause the computer system to perform any of the methods provided herein.

In yet another aspect of the present invention, to address the aforementioned problems, some embodiments provide a computer system. The computer system includes one or more processors, memory, and one or more programs. The one or more programs are stored in memory and configured to be executed by the one or more processors. The one or more programs include an operating system and instructions that when executed by the one or more processors cause the computer system to perform any of the methods provided herein.

### BRIEF DESCRIPTION OF THE DRAWINGS

FIG. 1 illustrates an example system for an Advanced Closed Loop (ACL) system for generating geothermal energy;

FIG. 2 illustrates an example method for quantifying the thermal conductivity of a subsurface formation;

FIG. 3 illustrates intermediate results of an example method for quantifying the thermal conductivity of a subsurface formation;

FIG. 4 illustrates a sideview of the thermal exchange section;

FIG. 5 illustrates a cross-section view of the thermal exchange section;

FIG. 6 illustrates a sideview of the thermal exchange section; and

FIG. 7 illustrates a cross-section of the thermal exchange section.

Like reference numerals refer to corresponding parts throughout the drawings.

### DETAILED DESCRIPTION OF EMBODIMENTS

Described below are methods, systems, and computer readable storage media that provide a manner of estimation of thermal conductivity of subsurface rock formations. These embodiments are designed to be of particular use for assessing subsurface rock formations for use in Advanced Closed Loop (ACL) systems used for generating geothermal energy. A key subsurface uncertainty in ACL is the efficiency of heat transfer of closed-loop wellbores with the surrounding rock. The efficiency of heat transfer is controlled by the thermal conductivity of the rock formation. However, there has not been a systematic method and workflow to characterize thermal conductivity along closed-loop wellbores which can extend horizontally for kilometers.

Thermal conductivity is the primary natural control on heat transfer in ACL. It isn't possible to directly measure the bulk thermal conductivity radially away from the wellbores (spatial scale of ˜100 m) and along the well bore (spatial scale of ˜1 km). The techniques described herein are used to upscale from thermal conductivity measured on special scales of centimeters to the bulk thermal conductivity of the reservoir (which is an annulus of 50-100 meters along the well bore).

Reference will now be made in detail to various embodiments, examples of which are illustrated in the accompanying drawings. In the following detailed description, numerous specific details are set forth in order to provide a thorough understanding of the present disclosure and the embodiments described herein. However, embodiments described herein may be practiced without these specific details. In other instances, well-known methods, procedures, components, and mechanical apparatus have not been described in detail so as not to unnecessarily obscure aspects of the embodiments.

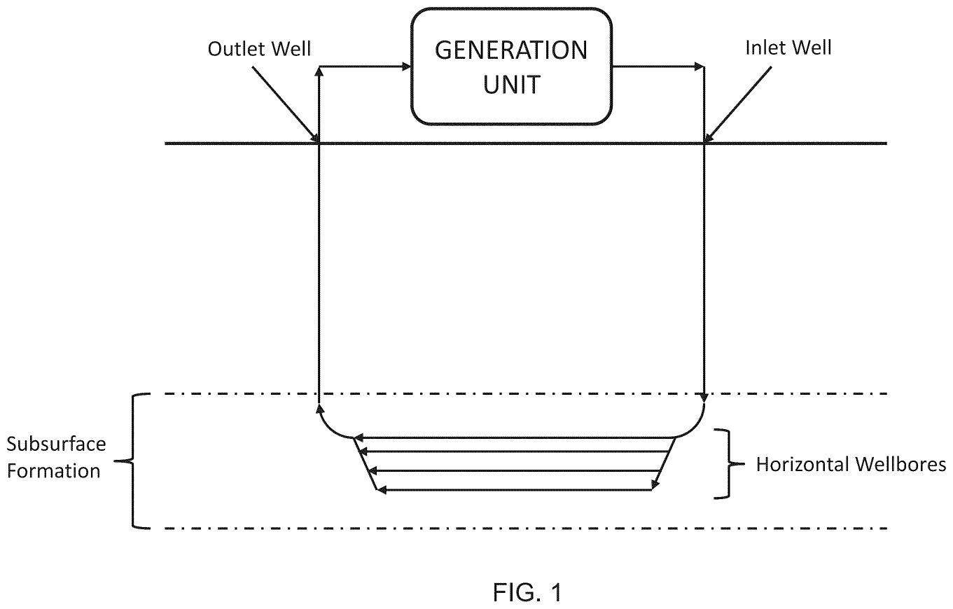

FIG. 1 is a simplified diagram of an Advanced Close Loop (ACL) system for geothermal energy. In this closed system, a vertical (or near vertical) wellbore is the inlet well, where the fluid of the closed system (called a working fluid) is injected into the subsurface. When the wellbore approaches the subsurface formation that has been selected based on its temperature and thermal properties, the wellbore turns to a near-horizontal track; multiple horizontal wellbores may be extended through the subsurface formation. The horizontal wellbores may be at different depths within the subsurface formation, spaced laterally from each other within the subsurface formation, or be located at both different depths and different lateral locations. When the working fluid traverses these horizontal wellbores, it is constrained to stay within the wellbores. If the horizontal wellbore is landed in impermeable and competent rock formations, open-hole completion is sufficient to constrain the working fluids within the wellbore. Otherwise, cased-hole completion may be needed to prevent fluid losses to surrounding formations and support the integrity of wellbore. The surrounding subsurface formation has thermal properties, including high thermal conductivity, that will heat the fluid. The heated fluid then moves up the substantially vertical outlet well and into the generation unit. Within the generation unit, the steam from the heated fluid turns turbines which produce electricity. The fluid will cool and then be reinjected into the inlet well and the process will repeat. This description is simplified; as is known to those of skill in the art, ACL systems may include additional or different components such as an Organic Rankine Cycle (ORC) component. The fluid used in the ACL may include water, water with additives, super critical carbon dioxide, and the like. There may be a primary working fluid that flows through the wellbores and a secondary working fluid within the generation unit. These examples are not meant to be limiting; the methods described herein are applicable to any ACL configuration.

The parts of the methods and systems of the present disclosure may be implemented by a system and/or in a system that may include one or more of a processor, an interface (e.g., bus, wireless interface), an electronic storage, a graphical display, and/or other components.

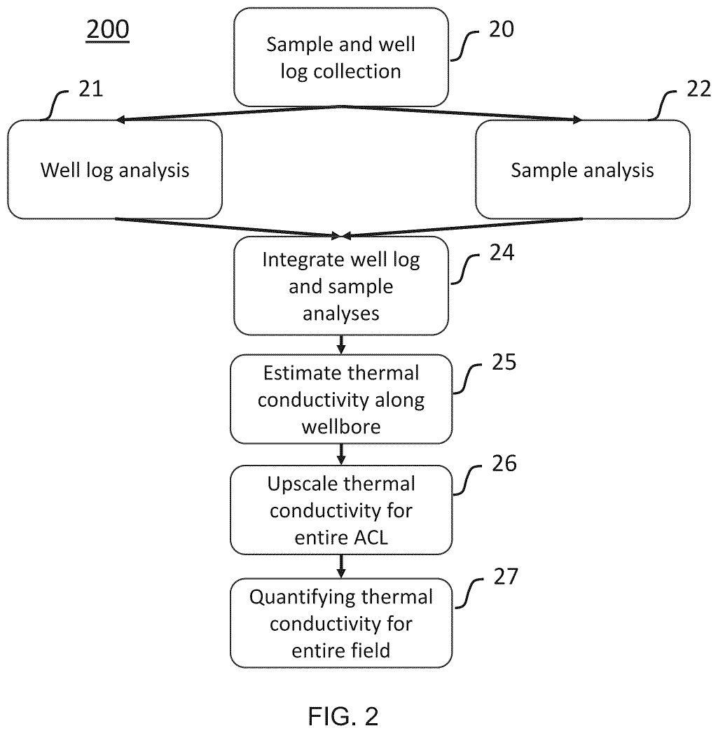

FIG. 2 illustrates an example process 200 for estimation of thermal conductivity of subsurface rock formations. At step 20, well logs are acquired continuously in the wellbore to record borehole temperature, pressure, and other near wellbore properties from a target geothermal formation and any subsurface formations above. Well logging may be limited by various Health, Safety and Environment (HSE) considerations. Typical well logging tools are designed for oil and gas exploration. They are rated to function under certain temperature and pressure. The formation temperature of the geothermal formations can be considerable higher than the temperature rating of typical well logging tools. Certain measures, such as mud circulation, can be used to reduce the wellbore temperature below the formation temperature, and in certain cases, also below the temperature rating of well logging tools. Another consideration is the potential environmental impact in the case that well logging tools, such neutron porosity and bulk density tools, are lost in hole in environmentally sensitive areas. The estimation of formation temperature, the borehole temperature controlling mechanism, and HSE considerations, are critical in designing well log acquisition programs based on logging-while-drilling (LWD) or wireline logging programs in vertical and horizontal sections. The logging program should be designed to acquire as much data as possible for the determination of rock mineralogy and fabric, porosity, and fluid properties, all of which are contributing to the estimation of thermal conductivity. Depending on the temperature and HSE limitations, and the complexity of the mineralogy, different well logging suites can be designed to acquire necessary parameters for thermal conductivity:
basic logging suites (gamma ray, neutron porosity, bulk density, resistivity)
   borehole temperature and pressure
   basic lithology
   porosity
   fluid composition and properties
advanced logging suites (spectral gamma ray, nuclear spectroscopy logs, borehole imaging, nuclear magnetic resonance, acoustic)
   nuclear spectroscopy logs for improved mineralogy (Wang et al., 2019)
   borehole imaging logs for fracture characterization
   nuclear magnetic resonance for permeability
sourceless logging suites (no nuclear source in bottom hole assembly)
   gamma ray or spectral gamma ray for lithology
   sourceless density for porosity (Simon et al., 2018)
   acoustic or NMR for porosity
   resistivity for fluid property and clay content
      The combination can be (1) basic logging suites, (2) basic and advanced logging suites, or (3) sourceless logging suites.

Also at step 20, core samples may be taken from selected interval in the case of whole core, or selected depth points in the case of rotary sidewall cores (RSWC). Laboratory measurements on core samples may include porosity, grain density, electrical resistivity, elemental composition, mineralogy, thermal conductivity, and rock mechanical properties. The core measurements are more robust and representative for subsurface formations. However, it often takes days or weeks for sample preparation and measurement, thus won't be able to use for making real-time decisions. Cuttings samples can be collected at the surface from liquid drilling fluid by shale shakers, at appropriate sampling intervals and sampling frequencies. Cuttings are produced as the rock is broken by the drill bit and carried to the surface by circulating up the drilling fluid. Cuttings samples can be collected at the surface from liquid drilling fluid by shale shakers, at appropriate sampling intervals and sampling frequencies. Cuttings are broken rock pieces therefore it is only a partial representative of subsurface rock formations. Such mechanical change does not impact the elemental composition or mineralogy. In addition, elemental composition on cuttings can be measured at the wellsite, providing timely description of the subsurface formation for deciding the landing point. It is a critically important to estimate thermal conductivity at the wellsite in order to provide timely inputs for real-time wellsite operations.

At step 21, well log analysis is performed. This may include interpreting acquired well logs for unique mineralogy and pore-filling fluids, as well as porosity, permeability, and rock mechanical properties for geothermal fields. Unlike the typical interpretation in the oil and gas exploration (Wang et al., 2019), petrophysical interpretation in geothermal fields is dealing with different type of rocks and log response, and often with only limited logging suites available. An embodiment of this step uses the following workflow: (1) identify minerals existing in the geothermal field based on existing core measurement or cuttings analysis from exploration wells, or from outcrop characterization, (2) select a group of representative minerals to be included in the petrophysical interpretation based on mineral abundance and its impact on log response, (3) determine the log response of representative minerals with existing log response database, forward modeling, or cross calibration with advanced well logs such as nuclear spectroscopy logs, (4) assuming an initial volumetric composition of the rock and fluids, predict log response of the rock from volumetric weighted average of the log response of each mineral and fluid composition, (5) compare the predicted and measured log response and calculate their difference, (6) by varying the volumetric composition of the rock and fluids, minimize the difference between the predicted and measured log response. The volumetric composition of the rock and fluids that lead to the best match of predicted and measured log response is the result of petrophysical interpretation.

At step 22, the method performs wellsite and laboratory measurement of core and cuttings samples. Several measurements can be performed based on cuttings samples at the wellsite, including basic description of lithology and fluid contents through microscopic examination of drilling cuttings, as well as advanced measurements such as elemental composition using portable X-ray Fluorescence (XRF) (Marsala et al., 2011), and porosity using Nuclear Magnetic Resonance (NMR) desktop instruments. Once samples reach laboratory, more accurate measurements are available including: (1) elemental composition using laboratory XRF and inductively coupled plasma (ICP) techniques (Marsala et al., 2011), (2) mineralogy using X-ray diffraction (XRD) (McCarty et al., 2015), Fourier-transform infrared spectroscopy (FTIR) (Craddock et al., 2016), and high-field NMR (Wang et al., 2022) (3) porosity and grain density using Routine Core Analysis, (4) porosity and pore size distribution using gas adsorption and NMR, (4) rock fabric using microscopy, (5) thermal conductivity using transient plane source (TPS) or optical thermal conductivity scanning (TCS). Rock mechanical properties can be performed on the core samples for the rock strength. This step may include collecting rock samples at certain depth intervals or at depth intervals of chosen spacing and sampling rate (i.e. every 10 or 30 ft); determining rock sample properties at the wellsite, such as porosity, elemental composition, lithology; determining rock sample properties in the laboratory, such as grain density, porosity, elemental composition, mineralogy, rock strength; and generating rock properties as function of depth that are equivalent to well logs with different sampling frequency

The analysis performed in step 22 means that the method can potentially rely on cuttings XRF that are available at the wellsite, plus an in-depth petrophysical analysis, to estimate thermal conductivity for making real-time operational decisions rather than having to wait for detailed lab measurement.

At step 24, the well log and sample analyses are integrated to determine mineralogy for geothermal fields, as well as porosity, saturation, and permeability if applicable. This may include depth shifting from core depth to well logs, harmonizing the core and well log data with the same sampling frequency using interpolation or resampling techniques, adjusting the group of minerals to be determined using a combination of well logs and core/cutting analysis, defining the chemistry and log response for each new mineral that is not in existing database, and solving for mineralogy, porosity, saturation, and permeability. This step helps to refine the results from step 21.

At step 25, the method estimates thermal conductivity along the wellbore using one or more mixing laws. The input data are mineralogy, rock fabric, porosity, and fluid types. Then this step defines a thermal conductivity for each mineral and fluid composition as a starting parameter and applies one of the anisotropic equivalent media mixing laws, such as Voigt-Reuss-Hill averaging, Hashin-Shtrikman bounds, Self-Consistent Approximation, Differential Effective Medium, Kuster-Toksoz model, or others, to generate a continuous thermal conductivity. Temperature correction and fluid substitution are required to further improve the predicted thermal conductivity. This is because the thermal conductivity values of each mineral and fluid composition are determined near ambient condition, it is important to apply a temperature correction for predicted thermal conductivity to the formation temperature of the geothermal field. Porosity and fluids have significant impact on the thermal conductivity. It is important to substitute different fluid type and saturation in predicted thermal conductivity when comparing with measured thermal conductivity from the laboratory or thermal conductivity of subsurface formation, as the fluid saturation condition may be different.

There are several methods for measuring thermal conductivity in the lab. Common methods include transient plane source (TPS) methods, transient line source (TLS) method, the USGS steady-state divided-bar technique, and the heat flow meter (HFM) methods. In TPS method, a disc-shaped sensor is placed in contact with the surface of two cylindrical rock core samples separated from one core sample. The TPS method employs a double-sided hot disc sensor to apply a heat pulse of several seconds to a few minutes to the sample. Temperature of the sensor is monitored with time and data is regressed to simultaneously determine thermal conductivity, thermal diffusivity, and specific heat capacity of materials from a single measurement. The thermal conductivity is measured at different temperature and fluid saturation environment. Due to the temperature and pore-fluid dependence of thermal conductivity, it is important to measure sample thermal conductivity at or close to the actual temperature and fluid saturation condition of geothermal fields.

By comparing the predicted thermal conductivity against the measured thermal conductivity from the laboratory, the mixing law that provides the best match is then chosen. In addition, the method can iteratively adjust the thermal conductivity of each mineral and fluid composition to minimize the difference between log-predicted and lab-measured thermal conductivity. Finally, the method at this step applies the refined mineral parameters and mixing laws to the entire intervals with well log and core dataset.

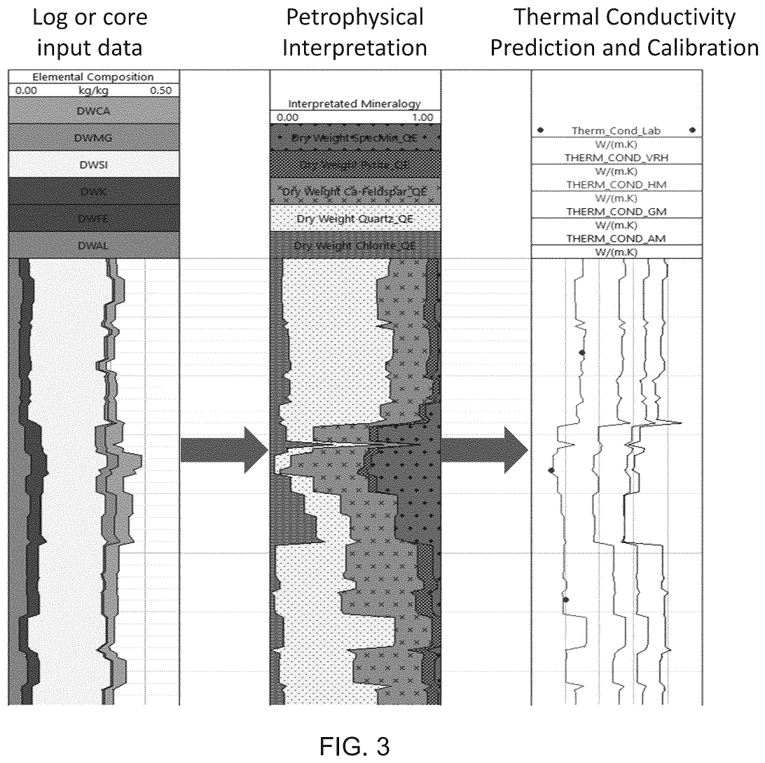

FIG. 3 shows input and intermediate results for steps 21-25. The left-hand track shows dry-weight elemental composition as an example of log or core input data from steps 21 and 22. Dry-weight elemental composition can be measured by downhole wireline logging or XRF cuttings analysis at the wellsite, both of which can be readily available to make the real-time operation decisions. These data are used as inputs for step 24 that produces advanced petrophysical interpretation as illustrated by dry-weight mineral composition in the center track. A depth-continuous interpretation of rock mineralogy is a critical step to link field measurements to thermal conductivity prediction in a timely fashion. The petrophysical interpretation are used as inputs for thermal conductivity prediction and calibration as described in step 25 and illustrated by the right-hand track. In the right-hand track, thermal conductivity is predicted from petrophysical interpretation using 4 anisotropic equivalent media mixing laws, namely Arithmetic Mean (AM), Geometric Mean (GM), Harmonic Mean (HM), and Voigt-Reuss-Hill (VRH) averaging. These predicted and continuous thermal conductivity logs are compared with lab-measured thermal conductivities values (shown as points). In this example, the Harmonic Mean method give the most accurate prediction compared to lab measured value. Continuous thermal conductivity log predicted by Harmonic Mean will be used for next steps to upscale thermal conductivity.

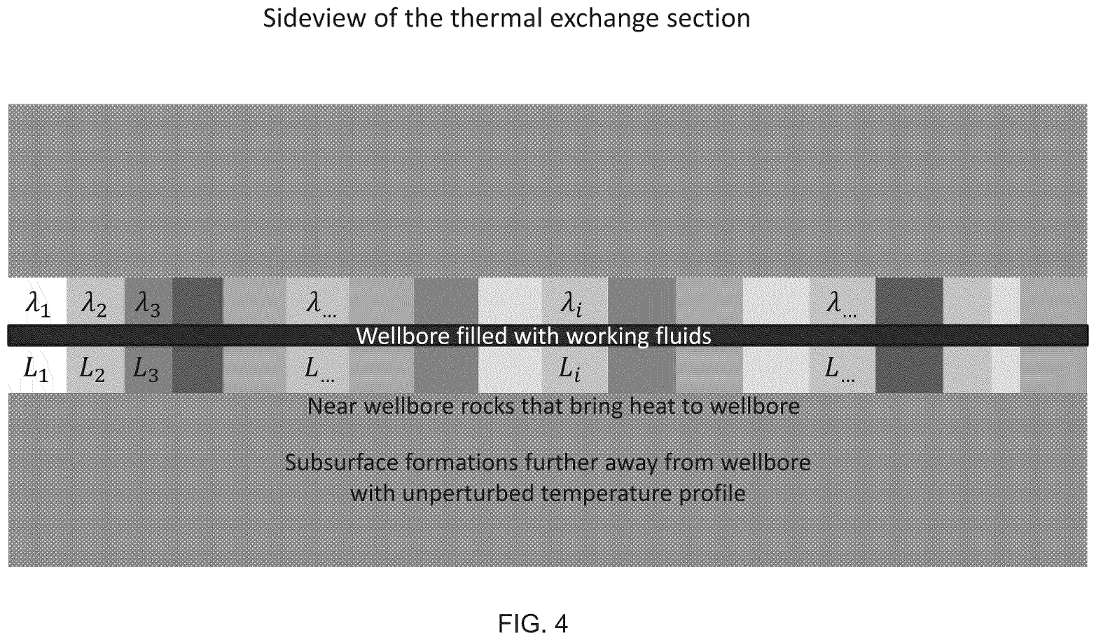

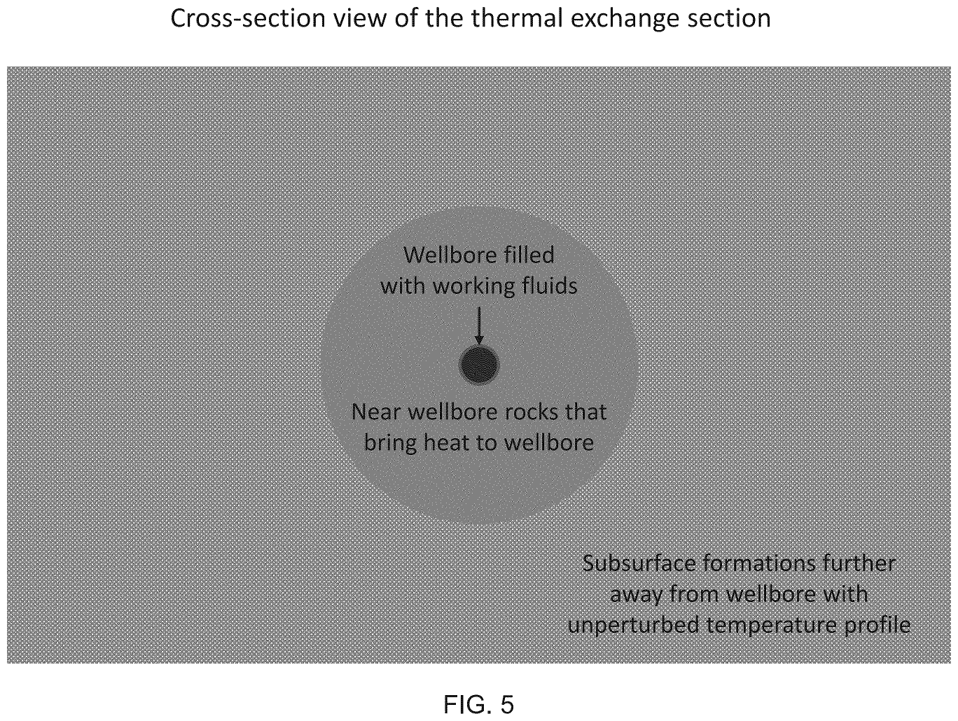

Once the method has estimated thermal conductivity along the wellbore at step 25, the method proceeds to step 26 to upscale the thermal conductivity estimation along the entire closed-loop system. To account for the variation of thermal conductivity along the wellbore, the near wellbore rocks that bring heat to wellbore working fluids are grouped by thermal conductivity values, At, of thickness Li along the direction of wellbore. This scenario is illustrated by FIG. 4 in sideview, and FIG. 5 in cross-section views of the thermal exchange sections, which is the open-hole sections that carry working fluids to extract heat from subsurface. This description is applicable for vertical, highly deviated, and horizontal wells. Assuming the thermal conductivity is isotropic in the radial direction of the wellbore, the effective bulk thermal conductivity integrated along the wellbore is given by:

$${λ}_{bulk} = \frac{\sum {L}_{i} \cdot {λ}_{i}}{\sum {L}_{i}}$$

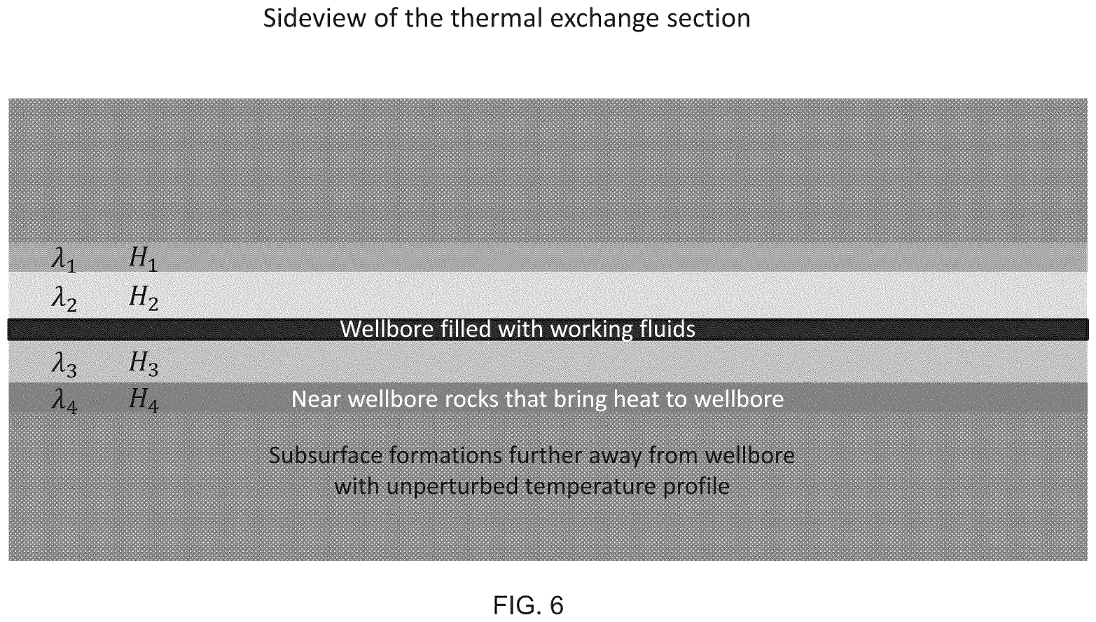

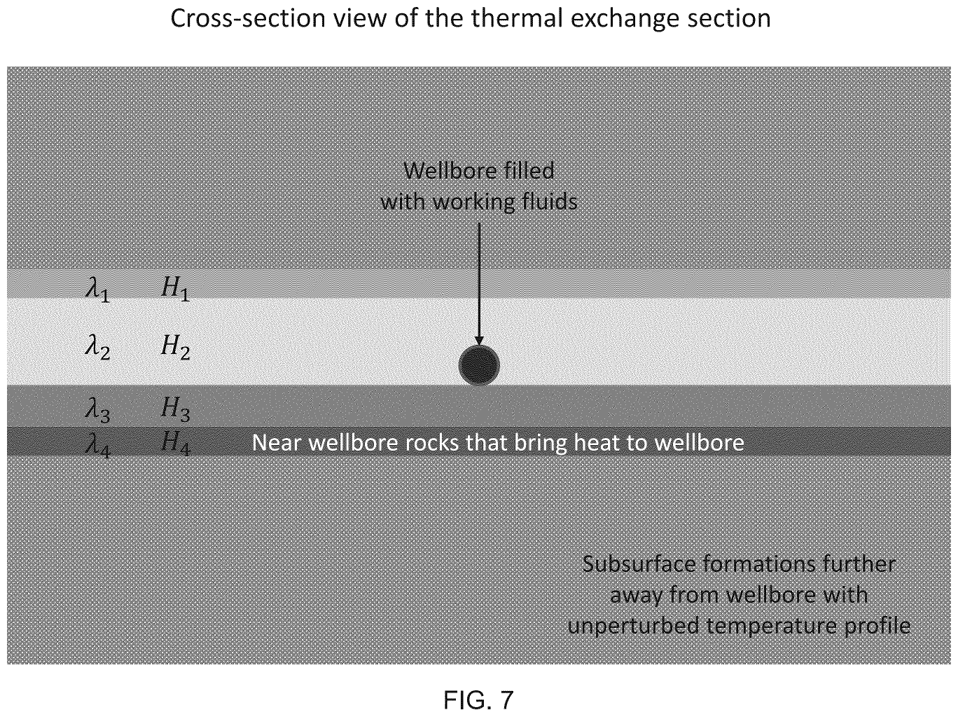

In the case that the wellbore is placed within a formation layer with relatively constant thermal conductivity but there is significant variation of among neighboring formation layers, as illustrated by FIG. 6 in sideview and FIG. 7 in cross section view, then the bulk thermal conductivity along vertical directions is given by:

$${λ}_{bulk} = \frac{\sum {H}_{i}}{\sum \frac{{H}_{i}}{{λ}_{i}}}$$

For more comprehensive calculation of bulk thermal conductivity, numerical modeling is required to combine the detailed thermal properties of near wellbore in the context of the temperature and thermal profile of the subsurface formation.

At step 27, the thermal conductivity estimation along the entire closed-loop system is extrapolated to the entire field or subsurface formation, away from the wellbore. This is done by upscaling the thermal conductivity to a broader depth interval and spatial area for geophysical analyses, correlating thermal conductivity to density and/or electrical resistivity for potential field analysis if applicable, identifying zones of geothermal interest with high temperature, high thermal conductivity, and stable rock chemistry and mechanical property, assessing possible vertical and lateral thermal resource numbers for exploration, and developing a well plan aiming for high thermal conductivity zones and low geological risks. This well plan is then implemented to run the asset and update catalogue.

While particular embodiments are described above, it will be understood it is not intended to limit the invention to these particular embodiments. On the contrary, the invention includes alternatives, modifications and equivalents that are within the spirit and scope of the appended claims. Numerous specific details are set forth in order to provide a thorough understanding of the subject matter presented herein. But it will be apparent to one of ordinary skill in the art that the subject matter may be practiced without these specific details. In other instances, well-known methods, procedures, components, and circuits have not been described in detail so as not to unnecessarily obscure aspects of the embodiments.

The terminology used in the description of the invention herein is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, operations, elements, and/or components, but do not preclude the presence or addition of one or more other features, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

Although some of the various drawings illustrate a number of logical stages in a particular order, stages that are not order dependent may be reordered and other stages may be combined or broken out. While some reordering or other groupings are specifically mentioned, others will be obvious to those of ordinary skill in the art and so do not present an exhaustive list of alternatives. Moreover, it should be recognized that the stages could be implemented in hardware, firmware, software or any combination thereof.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

### REFERENCES

### Addin En.Reflist

Craddock, P. R., Herron, M. M., & Herron, S. L. (2016). Quantitative mineral analysis of sedimentary formations using ftir spectroscopy. Paper SPWLA-2016-XX presented at the SPWLA 57th Annual Logging Symposium, Reykjavik, Iceland.
Horne, R. N. (1980). Design considerations of a down-hole coaxial geothermal heat exchanger. Paper presented at the Journal Name: Trans.-Geotherm. Resour. Counc.; (United States); Journal Volume: 4; Conference: Geothermal Resource Council annual meeting, Salt Lake City, UT, USA, 9 Sep. 1980, United States.
Marsala, A. F., Loermans, T., Shen, S., et al. (2012). Portable energy-dispersive x-ray fluorescence integrates mineralogy and chemostratigraphy into real-time formation evaluation. Petrophysics—The SPWLA Journal of Formation Evaluation and Reservoir Description, 53 (2), 102-109.
McCarty, D. K., Theologou, P. N., Fischer, T. B., et al. (2015). Mineral-chemistry quantification and petrophysical calibration for multimineral evaluations: A nonlinear approachpetrophysical calibration for multimineral evaluations. AAPG Bulletin, 99, 1371-1397.
Ramey, H. J., Jr. (1962). Wellbore heat transmission. Journal of Petroleum Technology, 14, 427-435.
Sass, J. H., Stone, C., Munroe, R. (1984) Thermal conductivity determinations on solid rock—a comparison between a steady-state divided-bar apparatus and a commercial transient line-source device, Journal of Volcanology and Geothermal Research, Volume 20, Issues 1-2, Pages 145-153
Simon, M., Tkabladze, A., Beekman, S., et al. (2018). A novel x-ray tool for true sourceless density logging. Petrophysics—The SPWLA Journal of Formation Evaluation and Reservoir Description, 59, 565-587.
Wang, H., Samano, L., Kelsch, K. D., et al. (2019). Pre-job planning based on nuclear modeling leads to successful downhole mineralogy determination in extremely challenging logging conditions. Paper presented at the SPWLA 60th Annual Logging Symposium.
Wang, H., Sun, B., Yang, Z., et al. (2022). Accurate rock mineral characterization with nuclear magnetic resonance. Petrophysics—The SPWLA Journal of Formation Evaluation and Reservoir Description, 63, 405-417.
White, M., Vasyliv, Y., Beckers, K., et al. (2024). Numerical investigation of closed-loop geothermal systems in deep geothermal reservoirs. Geothermics, 116, 102852.

What is claimed is:

1. A computer-implemented method of quantifying thermal conductivity of a subsurface formation, comprising:
a. obtaining rock samples and well logs from a well;
b. analyzing the well logs to generate a well log interpretation;
c. analyzing the rock samples to generate a rock sample interpretation;
d. integrating the well log interpretation and the rock sample interpretation to generate a combined interpretation;
e. estimating thermal conductivity along the well based on the combined interpretation using anisotropic equivalent media mixing laws;
f. upscaling the thermal conductivity along the well to quantify thermal conductivity for the well as part of an advanced closed loop geothermal system; and
g. quantifying the thermal conductivity of the subsurface formation based on the thermal conductivity for the well as part of the advanced closed loop geothermal system.

2. The method of claim 1 further comprising using the thermal conductivity of the subsurface formation to design well paths for the advanced closed loop geothermal system, drilling wells following the well paths, and building the advanced closed loop geothermal system.

3. The method of claim 1 wherein the well logs include basic logging suites, basic and advanced logging suites, or sourceless logging suites.

4. The method of claim 1 wherein the analyzing the well logs includes:
a. identifying minerals existing in the subsurface formation based on existing core measurement or cuttings analysis from exploration wells, or from outcrop characterization;
b. selecting a group of representative minerals to be included in the well log interpretation based on mineral abundance and its impact on well log response;
c. determining the well log response of each mineral in the group of representative minerals with existing log response database, forward modeling, or cross calibration with advanced well logs;
d. assuming a volumetric composition of rock and fluids and predicting the well log response of the rock from volumetric weighted average of the well log response of each mineral and fluid composition;
e. comparing the predicted well log response and measured well log response and calculating their difference;
f. varying the volumetric composition of the rock and fluids to minimize the difference between the predicted and measured log response; and
g. generating a well log interpretation based on the volumetric composition of the rock and fluids that lead to the best match of predicted and measured log response.

5. The method of claim 1 wherein the analyzing the rock samples includes:
a. obtaining the rock samples at specific depth intervals or at depth intervals of specific spacing and sampling rate;
b. determining wellsite rock sample properties of the rock samples at the well including at least one of porosity, elemental composition, lithology;
c. determining laboratory rock sample properties of the rock samples in a laboratory including at least one of grain density, porosity, elemental composition, mineralogy, rock strength; and
d. determining rock properties as function of depth that are equivalent to well logs with different sampling frequency by combining the wellsite rock sample properties and the laboratory rock sample properties to generate the rock sample interpretation.

6. The method of claim 1 wherein the integrating the well log interpretation and the rock sample interpretation includes depth shifting from core depth to depth in the well logs, harmonizing the rock samples and the well logs to a same sampling frequency using interpolation or resampling techniques, selecting a group of minerals to be determined using a combination of the well logs and rock samples, defining chemistry and log response for each new mineral that is not in existing database, and solving for at least one of mineralogy, porosity, saturation, and permeability.

7. The method of claim 1 wherein the estimating thermal conductivity along the well includes temperature correction and fluid substitution to match temperature and fluid content in the subsurface formation.
